(19) United States
(12) Patent Application Publication
(10) Pub. No.: US 2010/0191214 A1
(43) Pub. Date: Jul. 29, 2010

(54) MORE EASILY USABLE ADMINISTERING APPARATUS

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/644,291

(22) Filed: Dec. 22, 2009

Related U.S. Application Data
(63) Continuation of application No. PCT/CH2008/000269, filed on Jun. 13, 2008.

(30) Foreign Application Priority Data
Jun. 25, 2007 (CH) 1014/07

(51) Int. Cl. A61M 31/00

(52) U.S. Cl. 604/500; 604/131

(57) ABSTRACT

An administering device for liquid medicine including a basal rate selection switch for adjusting a basal rate, a bolus button encompassing an adjusting wheel for adjusting a bolus dose, and a trigger button for triggering administering of said dose, wherein the trigger button is centrally disposed in the adjusting wheel and the bolus button is arranged at an angle on the housing to make it easier to manipulate it, and wherein, to increase operational safety, a bolus is administered only when a specific sequence of states of the bolus button have been achieved and/or followed. In some embodiments, the bolus button may be additionally used for acknowledging alerts and/or, in some embodiments, a pillow-type, cushioning cover allows the device to be comfortably used also when sleeping. The invention encompasses a method of using an administering device, wherein during operation of the device, a basal rate selection switch is covered by a locking mechanism which locks a cartridge and an adapter for a catheter on a base unit.

### CROSS-REFERENCED RELATED APPLICATIONS

This application is a continuation of International Patent Application No. PCT/CH2008/000269 filed Jun. 13, 2008, which claims priority to Swiss Patent Application No. 1014/07 filed Jun. 25, 2007, the entire contents of both of which are incorporated herein by reference.

### BACKGROUND

The present invention relates to devices for injecting, infusing, administering, delivering and dispensing a substance, and to methods of making and using such devices. More particularly, it relates to a device or apparatus for administering a fluid product, e.g. a medicament in liquid form. In the following text, such a device or apparatus may be thought of and/or referred to as an administering apparatus.

Different diseases may require a medicament or therapeutic substance in a liquid form, such as an insulin preparation or a blood thinning medicament such as Heparin, to be administered to a patient over a relatively long period of time. Compact portable administering apparatuses which are always carried by the patient in the vicinity of the body are known for this purpose. On the one hand, the apparatus dispenses a predetermined basal rate of the medicament covering the usual basic need for the medicament in a continuous fashion or at regular intervals (e.g. every 20 minutes). On the other hand, provision can be made for the apparatus to be operated by the patient such that a bolus dose is dispensed if there is an increased need for the medicament. By way of example, provision can be made for a targeted dispensation of an insulin bolus after meals in the case of diabetic users.

Such apparatus can have a modular design, that is to say they can comprise a reusable base unit with a drive and a disposable cartridge with the medicament, wherein the cartridge can be connected to the base unit.

### SUMMARY

It is an object of the present invention to, on the one hand, simplify the operation of an administering apparatus when using it to administer a bolus and, on the other hand, make the apparatus and its use as safe and reliable as possible.

It is another object of the present invention to improve an apparatus for administering a fluid product so that it can also be comfortably worn by the patient at night without disturbing the patient's sleep.

It is another object of the present invention to prevent an inadvertent readjustment of the basal rate in a simple and reliable fashion.

Other objects of the present invention include to design the connection of base unit, cartridge and an adapter for attaching a catheter in a simple and secure fashion in the case of an administering apparatus with a modular design.

In one embodiment, the present invention comprises an administering device for a liquid medicine or therapeutic substance, the device comprising a basal rate selection switch for adjusting a basal rate, a bolus button encompassing an adjusting wheel for adjusting a bolus dose, and a trigger button for triggering administering of said dose, wherein the trigger button is centrally disposed in the adjusting wheel and the bolus button is arranged at an angle on the housing to make it easier to manipulate it, and wherein, to increase operational safety, a bolus is administered only when a specific sequence of states of the bolus button have been achieved and/or followed. In some embodiments, the bolus button may be additionally used for acknowledging alerts and/or, in some embodiments, a pillow-type, cushioning cover surrounds or covers the device to allow it, for example, to be comfortably used while the user is asleep. The invention encompasses a method of using an administering device, wherein during operation of the device, a basal rate selection switch is covered by a locking mechanism which locks a cartridge and an adapter for a catheter on a base unit.

In one embodiment, the present invention comprises an administering device for liquid medicine such as insulin, the device comprising a basal rate selection switch for adjusting a basal rate, and a bolus button encompassing an adjusting wheel for adjusting a bolus dose and a trigger button for triggering administering of said dose. The trigger button is centrally disposed in the adjusting wheel while the bolus button is altogether arranged at an angle on the housing to make it easier to manipulate. To increase operational safety, a bolus is administered only when a very specific sequence of states of the bolus button has been followed. In some embodiments, the bolus button is additionally used for acknowledging alerts. During operation, the basal rate selection switch is covered by a locking mechanism which locks a cartridge and an adapter for a catheter on a base unit. In some embodiments, a pillow-type cover allows the device to be comfortably used when the user is sleeping.

In accordance with a first aspect of the present invention, an apparatus for administering a fluid product, e.g a fluid medicament, is provided which comprises a product container and drive components for discharging the fluid product from the product container. In some preferred embodiments, the drive components are electrically operable. The apparatus is designed to, on the one hand, administer a basal rate quasi-continuously (that is to say either continuously or at predetermined intervals which ensure an even supply of the medicament to the patient) and, on the other hand, permit the additional targeted administration of a bolus dose. To this end, the apparatus comprises an adjustment device for setting or selecting the bolus dose and a trigger device for triggering the administration of the bolus dose; in some preferred embodiments, both the adjustment and trigger devices operate electrically, that is to say have corresponding electrical components. The adjustment device comprises an adjustment element, e.g. an adjustment wheel/rotary wheel or a rotary knob, which can rotate about a rotational axis and is used to operate a rotary switch, and the trigger device comprises a central trigger button which can be pressed in along the rotational axis and which is arranged concentrically with the adjustment element, which trigger button is used to actuate one or more electrical switches.

If a product container arranged in the apparatus has an elongate form, it thereby defines a longitudinal axis of the apparatus. The rotational axis of the adjustment element then extends at an angle to the longitudinal axis which does not equal 0, 90, 180 or 270 degrees so that it is more accessible from different directions. In some preferred embodiments, the angle between the rotational axis and the longitudinal axis is between approximately 30 degrees and approximately 60 degrees, in some preferred embodiments the angle is approximately 45 degrees.

If the apparatus has a substantially flat basic shape which defines a central apparatus plane, the rotational axis extends in the apparatus plane or parallel thereto. Combined with the abovementioned angular range, this ensures optimal accessibility and readability of the adjustment device.

In some preferred embodiments, when administering a bolus, an apparatus in accordance with the present invention only permits a predetermined sequence of states to avoid erroneous or unintended operations. To this end, the adjustment element can be rotated between an initialization position and a plurality of dose setting positions. The trigger element can be displaced along the rotational axis at least between an extended position and a retracted position. A bolus is only administered if the following sequence of states was passed through:

an initialization state in which the adjustment element assumes the initialization position thereof and the trigger element is extended into the extended position;

a bolus setting state in which the adjustment element assumes one of the dose setting positions thereof and in which the trigger element assumes the extended position thereof; and

a bolus trigger state in which the adjustment element assumes the last-mentioned dose setting position and in which the trigger element was inserted such that it assumes the retracted position.

Thus, the trigger element can only be moved into the extended position as a result of the adjustment element being rotated into the initialization position. Thus, the bolus can only be administered if the adjustment element is rotated into this position and the dose is subsequently set.

In some preferred embodiments, the trigger element is spring loaded toward the extended position along the rotational axis to thus afford the possibility of an automated extension of the trigger element when the adjustment element is in the initialization position.

In some embodiments, to prevent the trigger element from returning to the extended position after triggering the bolus, the trigger element has at least one latching element which, in the dose setting positions of the adjustment element, interacts with a support element arranged such that it is fixed to the housing in at least the axial direction such that it can only be moved in the direction of the retracted position from the extended position and latches with the support element in the retracted position. The latching element, and hence the trigger element, is released in respect of an axial displacement along the rotational axis from the retracted and into the extended position in the initialization position of the adjustment element. To this end, the support element can be an annular segment-shaped element which is fixed to the housing and discontinuous such that the latching element detaches from the support element in the initialization position.

In some embodiments, the trigger device can be designed to be additionally pushed against an elastic elastic force into a pushed-in position beyond the retracted position, and to actuate a further electrical contact in this position. As a result of this, the apparatus can be designed to only trigger a bolus administration once the trigger device was moved beyond the retracted position into the pushed-in position in the bolus trigger state and held in this position for a predetermined period of time against the elastic force.

In some embodiments, the trigger device can be used to acknowledge warnings or alarms provided by the apparatus. To this end, the apparatus comprises a warning device which is designed to monitor the operational state of the apparatus and to emit a signal, e.g. an acoustic and/or optical and/or vibratory signal or warning, in the case of an abnormal operational state. The trigger device then interacts with the warning device such that warning signals can be acknowledged to the warning device by pushing the trigger device from the retracted position into the pushed-in position, that is to say the warning device correspondingly changes the state thereof and possibly discontinues or interrupts the emission of the warning signals.

In accordance with a further aspect of the present invention, an apparatus for administering a fluid product, e.g. a fluid medicament, is provided which comprises a product container and drive components for discharging the fluid product from the product container. The apparatus is usually carried on the person with a belt or strap. To enable a patient to also be able to comfortably wear the apparatus on the body at night, without the apparatus disturbing the patient's sleep, provision is made for a cushion-like sleeve for use with the administering apparatus, which sleeve is made of a cushioning material and is suitable for completely containing the apparatus.

In some embodiments, the sleeve has an opening at an end face through which the apparatus can be inserted into the sleeve. If the apparatus has a display for displaying a set basal rate, it is advantageous in terms of operational safety for the display to be arranged on the apparatus such that the display can be read through the opening in a state in which the apparatus is inserted into the sleeve.

Typically, a catheter may be connectable to an apparatus in accordance with the present invention. The catheter then, in a state in which the apparatus is inserted into the sleeve, points from the apparatus, directly in the direction of the opening such that the catheter is not bent or kinked. The display for the basal rate is then arranged on the same side as the catheter, that is to say on that side of the apparatus which points to the opening in this state, e.g. on a short end face of an elongate apparatus with a flat design.

In some preferred embodiments, the sleeve has an opening which can, at least in part, e.g. centrally, be closed by a hook and loop fastener. However, other suitable closure or securing devices and/or methods may be used, e.g. snap fastener, a zip fastener, etc.

In some preferred embodiments, the thickness of the sleeve suffices to ensure a good cushioning effect. To this end, it has e.g. a thickness of at least 5 mm in all directions. In some preferred embodiments, the sleeve comprises a foamed material, which may be surrounded by a skin-friendly tissue, at least toward the outside.

In accordance with a further aspect of the present invention, an apparatus for administering a fluid product, e.g. a fluid medicament, comprises:

a base unit with a drive device and an adjustment device for setting a basal rate to be administered, which adjustment device interacts with a control unit for the drive device, e.g. in the case of an electronically controlled device; and

a locking device for locking or connecting to the base unit a replaceable cartridge with a product container for connection to an infusion set or a catheter, wherein the cartridge which can be connected to the base unit in a detachable fashion and/or by an adapter which can be connected to the base unit in a detachable fashion, wherein the locking device is designed to be moved between a locking position in which the cartridge and/or the adapter is/are locked to the base unit, and a release position in which the cartridge and/or the adapter can be removed from the base unit. To prevent an inadvertent adjustment of the basal rate, the locking device is designed such that it permits the basal rate to be set in the release position, while it prevents the basal rate from being set in the locking position. To this end, the locking device can at least partly cover the adjustment device in the locking position or block the adjustment device for the basal rate.

In some embodiments, the base unit has a display device for the set basal rate.

The display device can then be read in both the locking position and in the release position.

In one embodiment, the locking device comprises a flat bolt arranged on the base unit in a displaceable fashion, wherein the adjustment device and the display device are arranged on the base unit such that the bolt can be displaced over the adjustment device and the display device. The bolt has a recess or opening which uncovers both the display device and the adjustment device in the release position, but which only uncovers the display device in the locking position.

In one preferred embodiment, the adjustment device can be designed as follows: it comprises a rotary switch which can only be operated using a tool such as a screwdriver, which switch is on the circumference thereof provided with a scale which can be rotated with the rotary switch or the operating element thereof. Together with a marking fixed to the housing, e.g. with a window uncovering part of the scale, the scale forms a display device for the set basal rate.

In accordance with a further aspect of the invention, an apparatus for administering a fluid product, e.g. a fluid medicament, is provided, which apparatus is of modular design and comprises:

a base unit (a “reusable” module) with a drive device;

a replaceable cartridge (a “disposable” module) with a product container which can be connected to the base unit in a detachable fashion; and

an adapter which can be connected to the base unit in a detachable fashion for connecting an infusion set or a catheter.

In some embodiments, the apparatus has a locking device to lock both the cartridge and the adapter to the base unit. The locking device is designed to be moved between a locking position, in which both the cartridge and the adapter are locked to the base unit, a partly locking position, in which only the cartridge is locked to the base unit, and a release position, in which both the cartridge and the adapter can be removed from the base unit. This can achieve a locking of three components using a single device, e.g. a single bolt element. in some embodiments, the locking device at the same time is also used to prevent an adjustment of the basal rate in at least the first locking position, as described above.

in some preferred embodiments, the locking device is spring loaded from the partly locking position into the locking position and designed such that the locking device can be moved from the locking position into the partly locking position by the adapter when the adapter is connected to the cartridge, to then return to the locking position on its own accord after connecting the adapter to the cartridge and to latch to the adapter. This makes it possible for the adapter to latch to the base unit on its own accord while the cartridge remains locked to the base unit.

In some preferred embodiments, the locking device also comprises an operating element in the form of a flat bolt which is arranged on the base unit in a displaceable fashion. This bolt or a differently designed operating element is arranged on an end face of the base unit (e.g. the base unit is flat and elongate), which end face, in a state in which the adapter is locked to the base unit, points in the same direction as that side of the adapter to which a catheter is connected. This can easily achieve a locking of the adapter while operating is simplified at the same time because both the locking and the connection of the catheter are effected on the same side.

### BRIEF DESCRIPTION OF THE DRAWINGS

FIG. 1 is a schematic view of an embodiment of an administering apparatus in accordance with the present invention;

FIG. 2 is a schematic view of one embodiment of a sleeve in accordance with the present invention;

FIG. 3 is an illustration of how the administering apparatus of FIG. 1 is inserted into the sleeve of FIG. 2;

FIG. 4 is a view from the opening side of the sleeve with an administering apparatus held therein;

FIG. 5 is a schematic view of the administering apparatus with a detached cartridge and without a needle adapter;

FIG. 6 is a schematic view of one embodiment of an assembled administering apparatus in accordance with the present invention;

FIG. 7 is a schematic diagram of one embodiment of a bolt in accord with the present invention;

FIG. 8 is a schematic view of an embodiment of one embodiment of a bolus knob in a first position;

FIG. 9 is a schematic view of the bolus knob of FIG. 8 in a second position;

FIG. 10 was a central longitudinal section in the plane X-X through components of the bolus knob in a first orientation;

FIG. 11 is a schematic view from below of the components illustrated in FIG. 10;

FIG. 12 is a central longitudinal section in the plane XII-XII through the components of FIG. 10 in a second orientation;

FIG. 13 is a schematic view from below of the components illustrated in FIG. 12; and

FIG. 14 is a schematic plan view of one embodiment of a contact arrangement.

### DETAILED DESCRIPTION

With regard to fastening, mounting, attaching or connecting components of the present invention, unless specifically described as otherwise, conventional mechanical fasteners and methods may be used. Other appropriate fastening or attachment methods include adhesives, welding and soldering, the latter particularly with regard to the electrical system of the invention, if any. In embodiments with electrical features or components, suitable electrical components and circuitry, wires, wireless components, chips, boards, microprocessors, inputs, outputs, displays, control components, etc. may be used. Generally, unless otherwise indicated, the materials for making embodiments of the invention and/or components thereof may be selected from appropriate materials such as metal, metallic alloys, ceramics, plastics, etc. Unless otherwise indicated specifically or by context, positional terms (e.g., up, down, front, rear, distal, proximal, etc.) are descriptive not limiting. Same reference numbers are used to denote same parts or components.

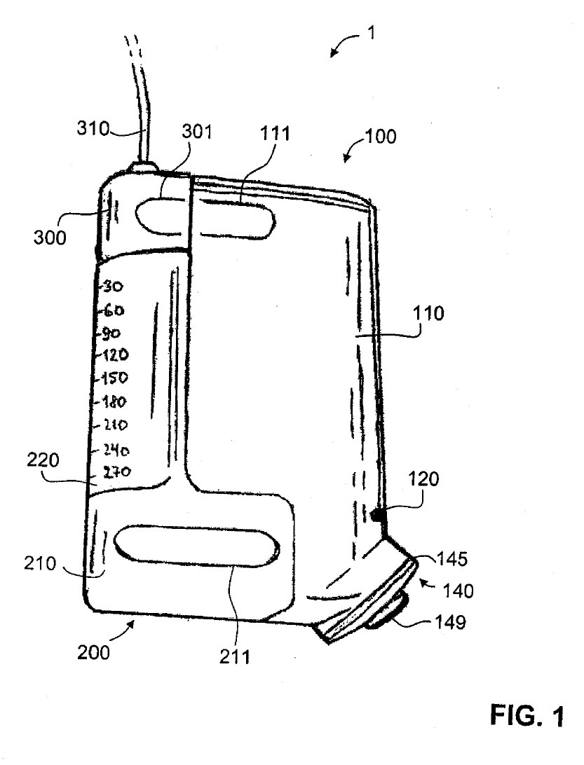

FIG. 1 shows a schematic view of one embodiment of an administering apparatus according to the present invention. The apparatus has a modular design and has a reusable base unit 100 (“reusable module”), a cartridge 200 (“disposable module”) connected detachably thereto and an adapter 300 affixed to the cartridge for connecting an infusion set with a catheter 310 and a cannula (not illustrated) for piercing the skin.

The base unit 100 has a housing 110 in which are arranged, inter alia, an electrical battery, control electronics, a motor, a transmission and a dog for transmitting the rotational motion generated by the motor and stepped-down by the transmission. An example of a suitably designed base unit is described in the international application PCT/CH2007/000113, dated Mar. 2, 2007, with reference being made thereto in respect of a possible more detailed embodiment of the base unit and the power transmission between base unit and cartridge. Two light emitting diodes 120, 121 are provided for displaying the current operational state of the apparatus and they can be seen particularly well in FIG. 3. A basal rate selection switch 160, visible in FIG. 5, is provided to set the basal rate; this switch will be described in more detail in the follow text. Moreover, a bolus knob 140 is present for setting and triggering a bolus dose; this knob, as the adjustment element, comprises an adjustment wheel 145 and a trigger button 149, acting as a trigger element, which can be pushed-in in the center along the rotational axis of the adjustment wheel 145.

The bolus knob 140 is arranged obliquely on the base unit for the purpose of improved operability. Here, the rotational axis of the adjustment wheel 145 runs at an angle of 45 degrees to the longitudinal axis of the apparatus, defined by the longitudinal axis of the carpule or ampoule, and is located in the central plane of the basically flat apparatus. As a result, the adjustment wheel is also very easily accessible for large fingers, even if the apparatus itself remains very compact. Additionally, it is easier to read the set dose from different directions.

The cartridge 200 has a housing 210 which houses a product container, for example in the form of a carpule, that is to say a cylindrical, ampoule-like container or vial which is generally composed of glass, with a piston which can be displaced therein. At the opposite end of the container to the piston, said container is sealed by a septum which can be pierced. Said container contains a liquid medicament to be administered, such as insulin, a blood-thinning drug, a painkiller or the like. The housing 210 is transparent, at least in the region of the container, and so the level of the container can be read through the housing wall. A scale 220 is affixed to the housing to ease the read out. Furthermore, components are housed in the cartridge, which are used to convert the drive motion generated by the base unit such that this leads to a discharge of the medicament. To this end, provision can be made for hydraulics to deflect and transmit the forces. Reference is again made to the international application PCT/CH2007/000113, dated Mar. 2, 2007, for an example of a suitably designed cartridge.

For the purpose of administering the medicament in the product container 250, the cartridge 200 is connected to the base unit 100. Thereupon the adapter 300 is pushed onto the cartridge; in FIG. 1 this is effected from the top. The adapter comprises a hollow needle which pierces the septum of the product container and thus connects the product container to the catheter 310. Surface structures (elements 111, 211 and 301) are formed on the base unit 100, the cartridge 200 and the needle adapter 300 for the purpose of improved handling and for preventing slippage. The cartridge 200 and the adapter 300 are fixed together onto the base unit 100 by a displaceable bolt (not visible in FIG. 1). The apparatus switches itself on automatically when the cartridge is connected to the base unit. After ventilating (priming) the catheter, an amount of the product, preselected by the basal rate selection switch, is dispensed during normal operation at predetermined intervals (e.g. three times an hour) and so the patient receives a predetermined basal rate in a substantially continuous fashion. The basal rate selection switch is usually set to a patient-specific value by a medical practitioner or a pharmacist, or possibly by the patient himself, when the apparatus is activated for the first time. If the patient requires an additional dose—a so-called bolus—said patient can set the desired dose by rotating the adjustment wheel 145 on the bolus knob 140 and trigger the administration of the bolus by pushing in the trigger button 149.

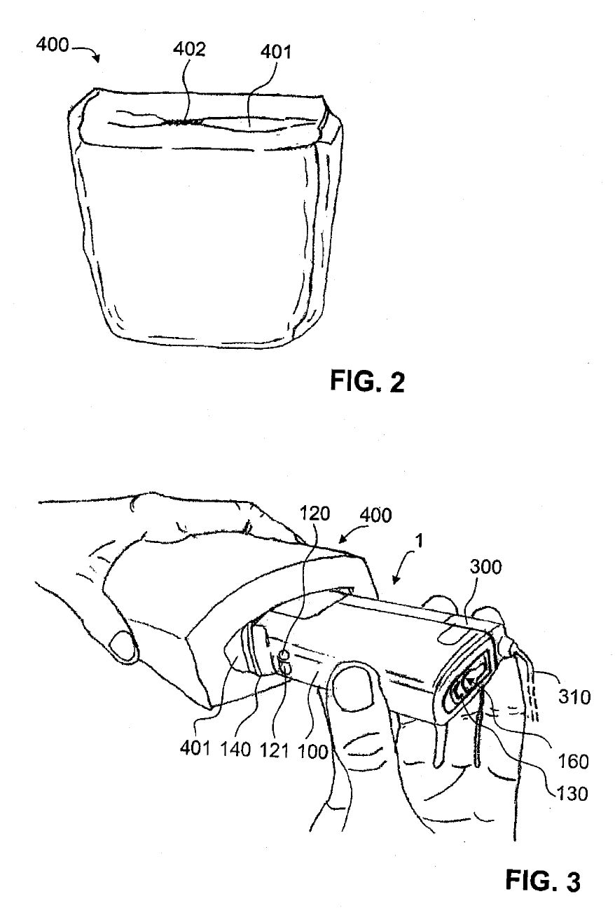

Such an administering apparatus is generally carried on the body over a relatively long period of time, both during the day and at night. During the day, it is, for example, attached to a belt by a belt clip or worn under the clothes in a special strap and/or a loop. So that the apparatus can also be comfortably kept near the body at night, a cushion-like bag or sleeve 400, illustrated in FIG. 2, is provided and the apparatus is inserted therein at night or during rest phases. The sleeve 400 has internal dimensions which approximately correspond to the external dimensions of the assembled administering apparatus. The administering apparatus can be inserted into the sleeve through an opening 401 in an end face of said sleeve, as illustrated in FIG. 3, and can be fixed in the sleeve using a hook and loop fastener 402 to prevent said apparatus from slipping out. Of course, the sleeve may be used at will, e.g., at times other than at night.

FIG. 3 also illustrates the orientation at which the administering apparatus is inserted into the sleeve. The adapter 300 is arranged at that end of the apparatus which points toward the opening 401 when the apparatus is completely inserted into the sleeve. As a result, the catheter 310 points directly toward the opening and can be guided out of the sleeve without deflection and without the risk of being bent or kinked. The bolt 130 also points in the same direction as the catheter.

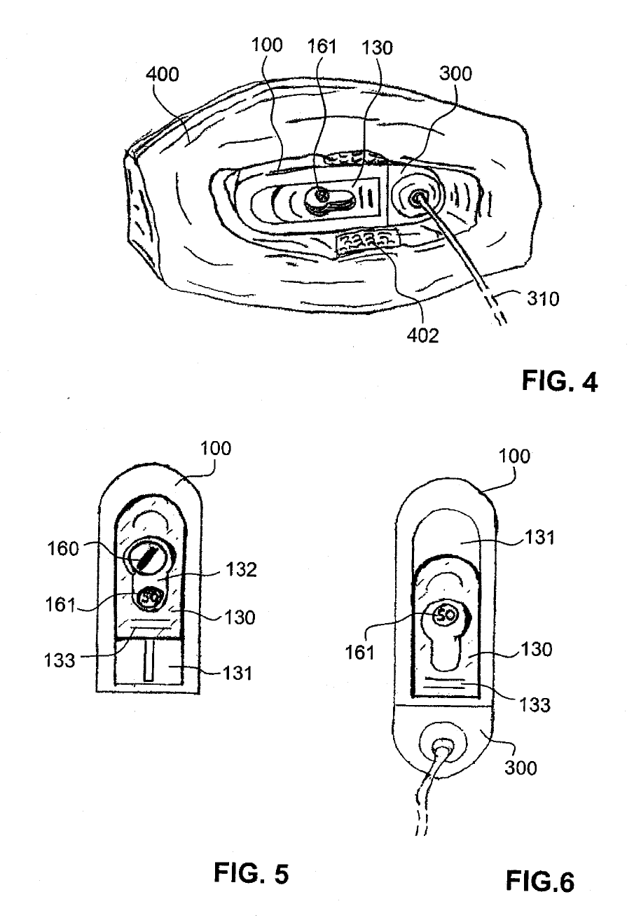

This bolt 130 can be seen particularly well in FIGS. 4 to 6. It has a flat operating element which is housed in a displaceable fashion in a displacement region formed by a flat recess in the end face housing wall, and by which the bolt 130 can be displaced between a release position illustrated in FIG. 5 and a locking position illustrated in FIG. 6. To prevent slippage, surface structures 133 are also formed on the operating element of the bolt 130. In the release position, the bolt allows unimpeded pulling off of the adapter 300 in that direction in which the catheter 310 points and unimpeded pulling off of the cartridge 200 in the opposite direction. By contrast, in the locking position, the bolt holds the cartridge 200 against the base unit. The bolt is spring loaded in this position and designed such that the adapter first slightly pushes the bolt in the direction of the release position when said adapter is pushed onto the cartridge in this situation. As soon as the adapter has reached the final position thereof, the bolt then latches into a corresponding recess on the adapter. As a result, the bolt 130 locks both the cartridge 200 and the adapter 300 to the base unit 100.

FIG. 7 illustrates, in a schematic fashion, how such a functioning of the bolt can be achieved in principle. In this embodiment, the bolt has two lugs 134, 135 which protrude through openings in the housing wall 110 of the base unit 100. The first lug 134 is used to lock the cartridge 200 (illustrated schematically), whereas the second lug 135 is used to lock the adapter 300. The first lug is longer than the second lug. The bolt is illustrated in the locking position. It is spring loaded by an elastic force 136. The cartridge 200 is already locked to the base unit as a result of the interlocking engagement with the lug 134, that is to say it can no longer be pulled off in the direction pointing downward in FIG. 7. Now the adapter 300 is pushed on in direction 137. In the process, as a result of the bevel of the second lug 135, said adapter slightly pushes the bolt against the direction 138 of the elastic force 136 into a partly locking position until the second lug 135 latches into the recess 139 of the adapter 300. Since the first lug 134 is longer than the second lug 135, the cartridge remains locked to the housing in the process, in the partly locking position as well, where the bolt is slightly pushed in the direction 135 by the adapter. It goes without saying that FIG. 7 is only a schematic schematic diagram and that many modifications of the principle illustrated therein are possible.

The bolt 130 is arranged on the housing 110 such that it covers the basal rate selection switch 160 in the locking position. This prevents the basal rate from inadvertently being changed when the apparatus is in operation. On the other hand, the bolt leaves the view onto a display 161 for the set basal rate uncovered in both the locking position and the release position. To this end, the operating element of the bolt has a keyhole-shaped elongate opening 132. It goes without saying that a different, e.g. likewise elongate or other, shape of the opening 132 is also possible instead of the keyhole shape illustrated here. In a simple fashion, the display 161 is implemented as a ring with numbers printed thereon and which ring is connected to the basal rate selection switch. When the switch is turned, the numbers on the ring rotate past a housing window and below the latter, the housing window uncovering the view onto respectively one number. In this embodiment, the basal rate selection switch is a rotary switch (coding switch) with a predetermined number (e.g. sixteen) of defined switch positions and it respectively latches in the switch positions in a noticeable fashion. Here, a switch position respectively corresponds to a specific basal rate, in the case of insulin, for example, the rate is in steps of two I.U. for between 12 and 22 I.U. per day, in steps of four I.U. for between 22 and 38 I.U., in steps of six I.U. for between 38 and 56 I.U. and in steps of eight I.U. for between 56 and 80 I.U. Thus, the scale of the rates to be administered relative to the switch position does not have to be linear. So as to prevent inadvertent actuation, in some embodiments the basal rate selection switch can only be adjusted with a suitable tool such as a screwdriver.

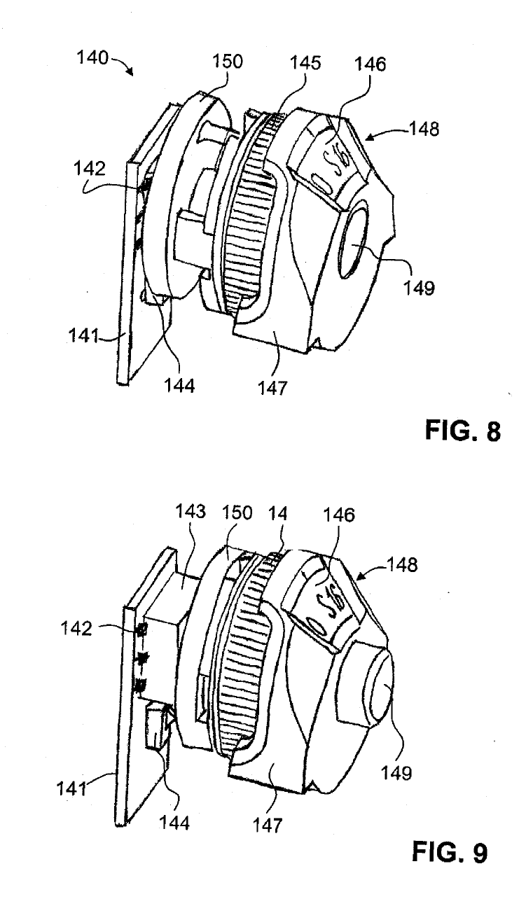

A bolus knob is provided for administering a bolus; the functioning of said knob will be explained in more detail with reference to FIGS. 8 to 14.

The bolus knob of FIGS. 8 and 9 has a base plate 141 provided with contacts 142. A multistage (e.g. twelve-stage) rotary switch 143 and an elastic switch pushbutton 144 with three switch positions are mounted on the base plate 141. The shaft of the rotary switch 143 is connected to a rotatable adjustment element in the form of an adjustment wheel 145, a scale 146 being affixed thereto, which scale can read through a window 148 in a cover 147 fixed to the housing. The adjustment wheel can be rotated between an initialization position (scale display “S”) and a plurality of dose setting positions (scale displays 0 to 16). A trigger button 149 is arranged concentrically with the adjustment wheel 145 and said button can be displaced axially along the rotational axis of the adjustment wheel 145 from an extended position illustrated in FIG. 8 to a retracted position illustrated in FIG. 7. Additionally, the trigger button can be pushed into a pushed-in position beyond the retracted position against an elastic force. The trigger button is connected to an annular transmission element 150. Depending on the position of trigger button, said transmission element pushes in the spring loaded pushbutton 144 by a certain amount: in the extended position of FIG. 8, the pushbutton is completely uncovered and assumes a first switch state. In the retracted position of FIG. 7, the pushbutton is pushed-in by a first amount and assumes a second switch state. In the pushed-in position therebeyond, the pushbutton assumes a third switch state. The position of the trigger button can thereby be determined electrically. It goes without saying that this can also be achieved by providing two separate switch elements for this purpose.

The bolus knob 140 and the control connected therewith are constructed such that a predetermined sequence of states has to be passed through for a bolus to be administered:

(a) In the supplied condition, the adjustment wheel is in the dose setting position “0” and the trigger button is in the retracted position. The trigger button cannot be moved from the retracted position into the extended position in a dose setting position.

(b) To bring the trigger button into the extended position, the adjustment wheel has to be brought into the initialization position “S”. The trigger button now jumps out as a result of an elastic force. This state can be referred to as the “initialization state”.

(c) A dose to be administered can now be set on the adjustment wheel by bringing the adjustment wheel into an appropriate dose setting position. The trigger button still assumes the extended position. This can be referred to as the “bolus setting state”.

(d) To administer the bolus, the trigger button is brought firstly into the retracted position and then into the pushed-in position therebeyond against the elastic force; it is kept in said pushed-in position until the apparatus acknowledges the administration of the bolus in an acoustic and/or optical fashion. This can be referred to as the “bolus trigger state”. Then, the trigger button is released and it remains in the retracted position. The adjustment wheel shows the now administered bolus by means of its position.

(e) To once again administer a bolus, steps (b) to (d) are once again carried out.

This sequence of steps ensures that a bolus cannot be administered inadvertently. Moreover, it affords the possibility of a positive operation acknowledgement in the case of the bolus administration and a later readout of the respectively last administered bolus.

In the process, the trigger button can also have additional functions. Thus, e.g., it can be used to acknowledge warnings or alarm signals from the apparatus if said apparatus determines an abnormal operational state, e.g. an occlusion, a battery that is too weak, etc.

The bolus knob as a whole can be used to trigger ventilation of the infusion set (“priming”) when starting up the apparatus after changing the cartridge. To this end, the apparatus awaits the above-described sequence to be performed once after the start up so that the amount of product required for priming is pumped through the infusion set.

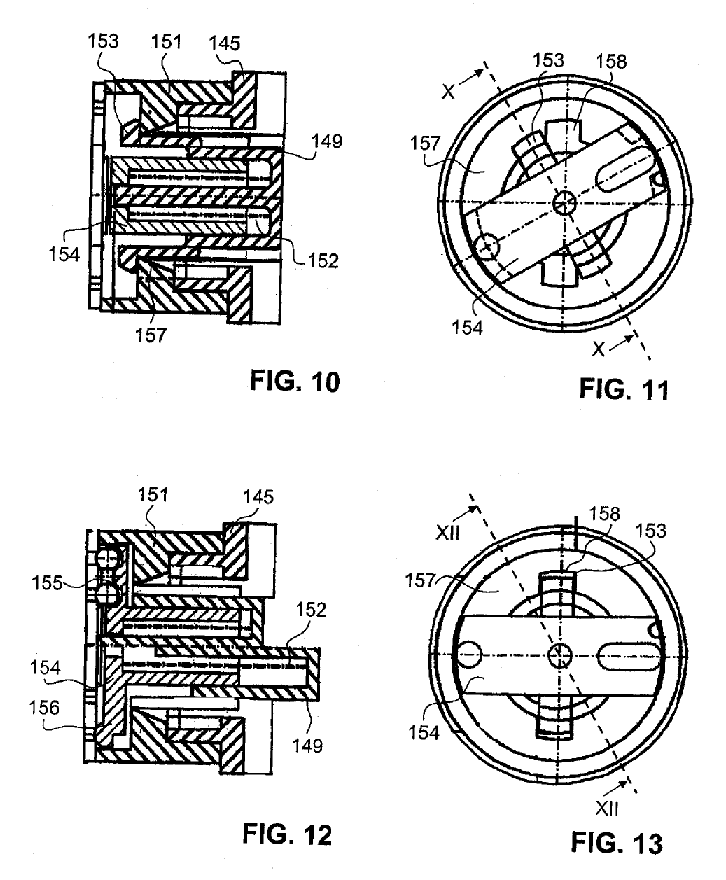

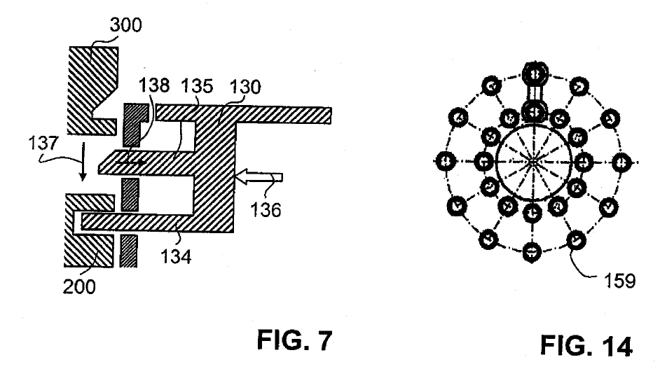

FIGS. 10 to 14 illustrate a mechanical implementation of a specified functionality of the bolus knob. Here, FIGS. 10 and 12 show a central longitudinal section through the relevant components of the bolus knob in two different rotational positions of the adjustment wheel 145: in FIG. 10, the adjustment wheel assumes one of the dose setting positions, while in FIG. 12 it assumes the initialization position “S” of FIGS. 8 and 9. Moreover, FIG. 12 illustrates two different positions of the trigger button 149 (upper half of the drawing: retracted position; lower half: extended position). FIGS. 11 and 13 show the corresponding components schematically from below and specify the sectional planes of FIGS. 10 and 12.

The adjustment wheel 145 is guided on a support element 151 fixed to the housing. The trigger button 149 is connected to the adjustment wheel 145 in an axially displaceable fashion and is fixed rotationally with respect to the adjustment wheel; that is to say it rotates with the adjustment wheel. The trigger button is axially spring loaded toward the outside in respect of a contact maker element 154, likewise connected to the adjustment wheel in a rotationally fixed and axially moveable fashion, by a pressure loaded helical spring 157. However, in the position of FIG. 10, two axially opposing latching hooks 153 prevent the trigger button from moving or jumping into the extended position as a result of this elastic force. The latching hooks 153 can be elastically bent radially toward the inside. As a result, the latching hooks can glide over an inner bevel face of an inwardly extending annular segment-shaped flange 157 of the support element 151 when the trigger element 149 moves from the extended position into the retracted position, whereas said hooks latch to the flange 157 in the retracted position.

By contrast, in the initialization position of FIG. 11, the latching hooks 153 come to rest in recesses 158 of the circular segment-shaped inner flange 157. As a result, the latching hooks 153 are no longer held axially, and the trigger button 149 can now jump from the retracted position shown in the upper half of FIG. 12 into the extended position shown in the lower half as a result of the elastic force of the spring 152.

In this extended position of the trigger button 149, the adjustment wheel can again be rotated into a dose setting position; however, unlike FIG. 10, the latching hooks 153 now come to rest to the right of the bevel surfaces of the flange 157. If the trigger button 149 is now pushed in, the latching hooks 153 glide over the bevel surfaces and, in the process, are elastically pushed in radially. As soon as the trigger button 149 reaches the retracted position, the latching hooks again jump radially outward. Hence, the position of FIG. 10 is reached again and the trigger button is once again held in the retracted position.

FIG. 14 illustrates how the contacts are arranged in the rotary switch 143. Respectively twelve contacts 159 are concentrically arranged on two different radii at regular angular distances. A contract bridge 155 of the contact maker element 154 is moved over the contacts 159 by the adjustment wheel and it thereby respectively connects two of the contacts. As a result of the elastic force of the spring 152, the contact maker element is pushed onto the contacts in the process and additionally latches into the slightly depressed contacts 159 with a latching lug 156.

It should be understood that other implementations of the described functionality of the bolus knob are also possible, and a large number of variants are also possible in respect of the design of the rest of the administering apparatus. Thus, e.g., the product container can also be designed as a container which can be compressed as a whole rather than being designed as a carpule, vial or ampoule. By way of example, the container can have a bellows-like side wall or simply be designed as a bag. The same holds true for the hydraulics reservoir. It is also conceivable for the transmission of the rotational motion onto or into the hydraulics reservoir to be effected by a flexible hydraulics container being “wrung out” by the proximal end thereof being rotated in respect of the distal end thereof. In a further variant, the hydraulic force transmission mentioned initially can be dispensed with.

Embodiments of the present invention, including preferred embodiments, have been presented for the purpose of illustration and description. They are not intended to be exhaustive or to limit the invention to the precise forms and steps disclosed. The embodiments were chosen and described to illustrate the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An administering device for administering a therapeutic substance, the device comprising:
a basal rate selection switch for adjusting a basal rate of administering;
a bolus button comprising an adjusting wheel for adjusting a bolus dose to be administered; and
a trigger button for triggering administering of said dose, wherein the trigger button is centrally disposed in the adjusting wheel and the bolus button is arranged at an angle on the device, and wherein, to increase operational safety, a bolus is administered only when a specific sequence of states of the bolus button have been achieved and/or followed.

2. The administering device according to claim 1, comprising a base unit, at least one of a cartridge and an adapter, and a lock for releaseably coupling the one of a cortridge and adapter to the base unit.

3. The administering device according to claim 2, wherein the lock removeably covers the basal rate selection switch.

4. The administering device according to claim 3, further comprising a cushioning cover substantially covering the device.

5. An apparatus for administering a fluid product, the apparatus comprising a product container and drive components for discharging the fluid product from the product container, wherein the apparatus is designed to administer a basal rate and permit an additional targeted administration of a bolus dose, the apparatus further comprising an adjustment device for setting the bolus dose and a trigger device for triggering the administration of the bolus dose, wherein the adjustment device comprises an adjustment element, rotatable about a rotational axis and the trigger device comprises a trigger button which can be pressed in along the rotational axis and which is arranged concentrically with the adjustment element.

6. The apparatus as clamed in claim 5, wherein the product container has an elongate form and thereby defines a longitudinal axis of the apparatus, and wherein the rotational axis is at an angle to the longitudinal axis other than 0, 90, 180 or 270 degrees.

7. The apparatus as claimed in claim 6, wherein the angle between the rotational axis and the longitudinal axis is between approximately 30 degrees and approximately 60 degrees.

8. The apparatus as claimed in claim 7, wherein the apparatus has a substantially flat basic shape which defines a central apparatus plane, and wherein the rotational axis is in the apparatus plane or parallel thereto.

9. The apparatus as claimed claim 5, wherein the adjustment element can be rotated between an initialization position and a plurality of dose setting positions, wherein the trigger element can be displaced along the rotational axis between at least an extended position and a retracted position, and wherein the apparatus is designed to only permit the administration of a bolus dose if the following sequence of states was passed through:
an initialization state in which the adjustment element assumes the initialization position thereof and the trigger element assumes the extended position thereof;
a bolus setting state in which the adjustment element assumes one of the dose setting positions thereof and in which the trigger element assumes the extended position thereof; and
a bolus trigger state in which the adjustment element assumes the mentioned dose setting position and in which the trigger element was inserted such that it assumes the retracted position.

10. The apparatus as claimed in claim 9, wherein the trigger element is spring loaded into the extended position along the rotational axis.

11. The apparatus as claimed in claim 10, wherein the trigger device comprises at least one latching element which interacts with the adjustment device such that, in one of the dose setting positions of the adjustment element, it interacts with a support element fixed to the housing in the axial direction such that it can only be moved in the direction of the retracted position from the extended position and latches with the support element in the retracted position, and that it is released in respect of an axial displacement along the rotational axis from the retracted and into the extended position in the initialization position of the adjustment element.

12. The apparatus as claimed in claim 11, wherein the support element comprises an annular segment-shaped element fixed to the housing and discontinuous such that the latching element detaches from the support element in the initialization position.

13. The apparatus as claimed in claim 12, wherein the trigger device can be additionally pushed against an elastic spring force into a pushed-in position beyond the retracted position.

14. The apparatus as claimed in claim 13, wherein the apparatus is designed to only trigger a bolus administration after the trigger device was pushed beyond the retracted position into the pushed-in position in the bolus trigger state and held in this position for a predetermined period of time against the elastic force.

15. The apparatus as claimed in claim 14, wherein the apparatus comprises a warning device designed to monitor the operational state of the apparatus and to emit warning signals in the case of an abnormal operational state, and wherein the trigger device interacts with the warning device such that warning signals can be acknowledged to the warning device by pushing the trigger device from the retracted position into the pushed-in position.

16. An apparatus for administering a a fluid medicament, wherein the apparatus comprises a product container and drive components for discharging the medicament from the product container, and a sleeve which is made of a cushioning material and is suitable for completely holding the apparatus so as to make it easier for a user to wear the apparatus.

17. The apparatus as claimed in claim 16, wherein the sleeve has an opening through which the apparatus can be inserted into the sleeve, wherein the apparatus has a display device for displaying a set basal rate, and wherein the display device is arranged on the apparatus such that the display device can be read through the opening in a state in which the apparatus is inserted into the sleeve.

18. The apparatus as claimed in claim 17, wherein the apparatus can be connected to a catheter such that the catheter, in a state in which the apparatus is inserted into the sleeve, points from the apparatus directly in the direction of the opening of the sleeve and such that the display device for the basal rate is arranged on a side of the apparatus which points to the opening in this state.

19. The apparatus as claimed in claim 18, wherein the sleeve defines an opening which can at least in part be closed by a hook and loop fastener.

20. The apparatus as claimed in claim 19, wherein the sleeve has a thickness of at least 5 mm.

21. The apparatus as claimed in claim 20, wherein the sleeve comprises a foam material.

22. An apparatus for administering a fluid product, comprising:
a base unit with a drive device and an adjustment device for setting a basal rate to be administered; and
a locking device for releaseably locking to the base unit at least one of a replaceable cartridge with a product container which can be connected to the base unit in a detachable fashion and an adapter for connecting an infusion set which can be connected to the base unit in a detachable fashion, wherein the locking device is moveable between a locking position, in which the at least one of the cartridge and adapter is/are locked to the base unit, and a release position, in which the at least one of the cartridge and the adapter can be removed from the base unit, wherein
the locking device permits the basal rate to be set in the release position and prevents the basal rate from being set in the locking position.

23. The apparatus as claimed in claim 22, wherein the locking device at least partly covers the adjustment device in the locking position.

24. The apparatus as claimed in claim 23, wherein the base unit further comprises a display device for the set basal rate, and wherein the display device is readable in both the locking position and in the release position.

25. The apparatus as claimed in claim 24, wherein the locking device comprises a flat bolt arranged on the base unit in a displaceable fashion, wherein the adjustment device and the display device are arranged such that the bolt can be displaced over the adjustment device and the display device, and wherein the bolt has an opening which uncovers both the display device and the adjustment device in the release position, but which only uncovers the display device in the locking position.

26. The apparatus as claimed in claim 25, wherein the adjustment device comprises a rotary switch having a circumference, a scale on the circumference which can be rotated with the rotary switch, which scale together with a marking fixed to the housing forms a display device for the set basal rate.

27. An apparatus for administering a fluid produc, comprising
a base unit with a drive device;
a replaceable cartridge with a product container which can be connected to the base unit in a detachable fashion; and
an adapter for connecting a catheter which can be connected to the cartridge in a detachable fashion; wherein the apparatus further comprises:
a locking device for locking both the cartridge and the adapter to the base unit, wherein the locking device is moveable among a locking position, in which both the cartridge and the adapter are locked to the base unit, a partly locked position, in which only the cartridge is locked to the base unit, and a release position, in which both the cartridge and the adapter can be removed from the base unit.

28. The apparatus as claimed in claim 27, wherein the locking device is spring loaded from the partly locked position into the locking position and is movable against an elastic force from the locking position into the partly locked position by the adapter when the adapter is connected to the cartridge, to then return to the locking position after connecting the adapter to the cartridge as a result of the elastic force and to latch to the adapter.

29. The apparatus as claimed in claim 28, wherein the locking device comprises an operating element in the form of a flat bolt arranged on the base unit in a displaceable fashion.

30. The apparatus as claimed in claim 29, wherein the locking device further comprises an operating element arranged on an end face of the base unit, which end face, in a state in which the adapter is locked to the base unit, points in the same direction as that side of the adapter to which the catheter can be connected.

31. A method of administering a therapuetic substance using an administering device, comprising the steps of:
selecting a basal rate of administering using a basal rate selection switch;
selecting a bolus dose to be administered using an adjusting wheel associated with a bolus button; and
triggering the administration of a selected bolus dose using a trigger button, wherein the trigger button is centrally disposed in the adjusting wheel and the bolus button is arranged at an angle on the device, and wherein, to increase operational safety, a selected bolus dose can be administered only when a specific sequence of states of the bolus button have been achieved and/or followed.
